(19) United States
(12) Patent Application Publication
(10) Pub. No.: US 2018/0037543 A1
(43) Pub. Date: Feb. 8, 2018

(54) METHOD FOR PRODUCING OXYSULPHIDIC AND FLUORINATED DERIVATIVES IN AN IONIC LIQUID MEDIUM

(71) Applicant: RHODIA OPERATIONS, Paris (FR)

(72) Inventor: François METZ, Irigny (FR)

(21) Appl. No.: 15/534,091

(22) PCT Filed: Dec. 7, 2015
(86) PCT No.: PCT/EP2015/078758

§ 371 Date: Oct. 11, 2017

(30) Foreign Application Priority Data
Dec. 9, 2014 (FR) 1462099

(51) Int. Cl. C07C 303/14; C07C 303/32; C07C 309/80; C07C 309/06; C07C 311/48; C07C 313/04; C07C 303/38

(52) U.S. Cl. CPC C07C 303/14; C07C 313/04; C07C 303/32; C07C 303/38; C07C 309/06; C07C 311/48; C07C 309/80

(57) ABSTRACT

The invention relates to a method for producing an oxysulphidic and fluorinated derivative in the form of a salt of formula (II) Ea-SO⁻Q⁺ (II) comprising providing an ionic liquid compound of formula (I) in the liquid state Ea-SOO⁻Cr (II)—Ea representing the fluorine atom or a group having between 1 and 10 carbon atoms selected from fluoroalkyls, perfluoroalkyls and fluoroalkenyls; and—Q⁺ representing an onium cation, with a sulphur oxide, said ionic liquid compound of formula (I) representing at least 50 wt. % of the initial liquid reactive medium.

A subject matter of the present invention is a novel process for the preparation of oxysulfide and fluorinated derivatives by a sulfination reaction carried out in an ionic liquid medium performing the double role of solvent and reactant.

The invention is more particularly targeted at the preparation of perfluoroalkanesulfinic and -sulfonic acid salts and preferably trifluoromethanesulfinic and trifluoromethanesulfonic acid salts.

Perhaloalkanesulfonic acids, and more particularly trifluoromethanesulfonic acid, better known as “triflic acid”, are used as catalysts or as intermediates in organic synthesis.

A current route for the industrial synthesis of trifluoromethanesulfonic acid employs two mains stages. First, an alkali metal salt, generally the potassium salt, of trifluoromethanesulfinic acid is synthesized by a sulfination reaction starting from a trifluoromethanecarboxylic acid salt, in an organic aprotic solvent, typically N,N-dimethylformamide (DMF). Secondly, the trifluoromethanesulfinic acid salt is oxidized in aqueous medium, generally by aqueous hydrogen peroxide, to give a trifluoromethanesulfonic acid salt, which, after acidification, will give triflic acid. The preparation of perfluoromethanesulfinic acids in the salified form is, for example, described in the documents EP 0 735 023 and WO 2007/128893.

Even though this process is generally satisfactory, some elements might be improved. First, it would be desirable to limit the stages of organic medium/aqueous medium switching between the sulfination and oxidation reactions since these switching stages can be complex to carry out. In addition, the presence of water during the acidification stage is a disadvantage, and means must be employed to capture this residual water, typically the addition of sulfuric anhydride (SO₃). The addition of sulfuric anhydride to capture the residual water unfortunately results in the generation of a large amount of sulfuric effluents.

The present invention is targeted at providing a novel process for the preparation of oxysulfide and fluorinated derivatives which is in particular of use in the synthesis of trifluoromethanesulfonic acid and which does not exhibit the abovementioned disadvantages.

More specifically, the present invention relates, according to a first of its aspects, to a process for the preparation of an oxysulfide and fluorinated derivative in the form of a salt of formula (II):

Ea-SOO⁻Q⁺ (II)

comprising the operation in which an ionic liquid compound of formula (I) in the liquid state:

Ea-COO⁻Q⁺ (I)
Ea representing a fluorine atom or a group having from 1 to 10 carbon atoms selected from fluoroalkyls, perfluoroalkyls and fluoroalkenyls; and
Q⁺ representing an onium cation,

is brought together with a sulfur oxide,

said ionic liquid compound of formula (I) representing at least 50% by weight of the initial liquid reaction medium.

Surprisingly, the inventors have shown that the sulfination reaction could be carried out in an ionic liquid medium, performing the double role of solvent and reactant, with performance levels in terms of kinetics and of selectivity at least identical to those of a sulfination in an organic solvent medium, typically DMF.

Advantageously, the process according to the invention thus makes it possible to dispense with the use of an ancillary solvent, typically DMF, in order to carry out the sulfination.

Equally, as expanded on in the continuation of the text, it is possible according to the invention to carry out the oxidation, for example in order to access a trifluoromethanesulfonic acid salt, starting from the mixture of the oxysulfide and fluorinated derivative Ea-SOO⁻Q⁺ (II) resulting from the sulfination and of the unreacted ionic liquid compound Ea-COO⁻Q⁺ (I), which is liquid at the temperature of the oxidation reaction and which acts both as reactant and as solvent for the oxidation reaction.

Thus, the oxidation according to the invention can be carried out without requiring the addition of aqueous solvent.

Advantageously, the sulfination and oxidation stages according to the invention can be carried out successively and without an intermediate stage of switching of solvents (organic medium/aqueous medium), in particular within the same reactor.

The process of the invention advantageously makes possible a saving in time and energy and thus a reduction in the cost price, due to the reduction in the number of stages necessary to obtain the trifluoromethanesulfonate salt (and triflic acid), for example.

Moreover, linking the sulfination and oxidation stages according to the invention makes it possible to minimize the degradation of the reaction stream resulting from the sulfination, which may take place during the switching of solvents.

Thus, the implementation of the process of the invention makes it possible to improve the overall yield of the preparation of the trifluoromethanesulfonate salt (and triflic acid).

Finally, the process of the invention, by dispensing with the use of an aqueous solvent for the oxidation, makes it possible to access triflic acid of electronic quality, exhibiting a low content of sulfates, indeed even being devoid of sulfates.

Of course, the synthesis of triflic acid is given by way of illustration, the process of the invention being in no way limited just to the synthesis of a trifluoromethanesulfonate salt and to that of triflic acid.

Other characteristics, alternative forms and advantages of the process according to the invention will become more clearly apparent on reading the description and examples which will follow, given by way of nonlimiting illustration of the invention.

In the continuation of the text, the expressions “between . . . and . . .”, “ranging from . . . to . . .” and “varying from . . . to . . .” are equivalent and are intended to mean that the limits are included, unless otherwise mentioned.

As specified above, the process for the preparation of an oxysulfide and fluorinated derivative in the form of a salt of formula Ea-SOO⁻Q⁺ (II) according to the invention involves a sulfination reaction of an ionic liquid compound of formula Ea-COO⁻Q⁺ (I) with a sulfur oxide. The operation in which the ionic liquid compound of formula (I) and a sulfur oxide are brought together is carried out under conditions favorable to the formation of the derivative of formula (II).

The ionic liquid compound of formula (I) according to the invention acts both as reactant and as solvent for the sulfination reaction.

The initial reaction medium of the sulfination reaction according to the invention comprises said ionic liquid compound of formula (I) in the liquid state at the temperature of the reaction and optionally the sulfur oxide, according to whether the reaction is carried out batchwise, semibatchwise or continuously.

“Reaction medium” is understood to mean, within the meaning of the invention, the medium in which the chemical reaction in question takes place; in the present case the sulfination reaction. The reaction medium generally comprises the reaction solvent (in the present case the ionic liquid compound, which is also one of the reactants) and, depending on the progression of the reaction, the reactants and/or the products of the reaction. In addition, it can comprise additives and impurities.

The “initial” reaction medium is the medium employed in the reactor before reaction and formation of the desired product. The initial reaction medium can comprise one of the reactants (for example, semibatchwise) or all of the reactants (for example, batchwi se).

In the case of the present sulfination reaction, the ionic liquid compound of formula (I), playing the double role of solvent and reactant, represents at least 50% by weight of the initial liquid reaction medium.

In the specific case of the batchwise implementation of the sulfination reaction, the initial reaction medium comprises the ionic liquid compound and the whole of the amount of sulfur oxide. The ionic liquid compound of formula (I) can represent from 50% to 95% by weight of the initial reaction medium.

In the specific case of the semibatchwise implementation of the sulfination reaction, the sulfur oxide can be added continuously. The ionic liquid compound of formula (I) can thus represent from 50% to 100% by weight of the initial reaction medium.

Preferably, the ionic liquid compound of formula (I) represents at least 60% by weight, in particular at least 70% by weight and more particularly at least 80% by weight of the initial reaction medium.

Preferably, the reaction medium of the sulfination reaction according to the invention is devoid of solvent other than said ionic liquid compound.

“Solvent” is intended to denote, within the meaning of the invention, a compound which is liquid at its temperature of use and which is capable, due to its content in the reaction medium, of dissolving a reactant.

In particular, the reaction medium of the reaction is devoid of organic solvent, especially of organic solvent of amide type, such as

N,N-dimethylformamide (DMF), N-methylpyrrolidone (NMP) or

N,N-dimethylacetamide (DMAC).

According to a particularly preferred embodiment, the initial liquid reaction medium of the sulfination reaction according to the invention is formed of the ionic liquid compound of formula (I) in the liquid state at the temperature of the reaction and optionally of a sulfur oxide, according to whether the reaction is carried out batchwise, semibatchwise or continuously.

Generally, ionic liquids are liquids which essentially contain only ions. The expression “ionic liquid” denotes a salt having a melting point of less than or equal to 100° C.

Ionic liquids encompass in particular a group of ionic compounds which are liquid at ambient temperature (20° C.) and which are known as “Room-Temperature Ionic Liquids” (RTILs).

Thus, within the meaning of the invention, in accordance with the definition commonly recognised, the ionic liquid compound of formula (I) employed according to the process of the invention has a melting point of less than 100° C.

In particular, the ionic liquid compound of formula (I) has a melting point of less than 80° C., in particular of less than 60° C. and more particularly of less than 40° C.

According to a specific embodiment, the ionic liquid compound according to the invention has a melting point of less than 20° C. (room-temperature ionic liquid).

The ionic liquid compound of formula (I) according to the invention is thus in the liquid state under the conditions of the sulfination reaction.

As indicated above, Q⁺ represents an onium cation.

Onium cations are cations formed by the elements of Groups VB and VIB (as defined by the old European IUPAC system according to the Periodic Table of the Elements) with three or four hydrocarbon chains. The Group VB comprises the N, P, As, Sb and Bi atoms. The Group VIB comprises the O, S, Se, Te and Po atoms. The onium cation can in particular be a cation formed by an atom selected from the group consisting of N, P, O and S, more preferably N and P, with three or four hydrocarbon chains.

The onium cation Q⁺ can be selected from:
Heterocyclic onium cations; in particular those selected from the group consisting of:

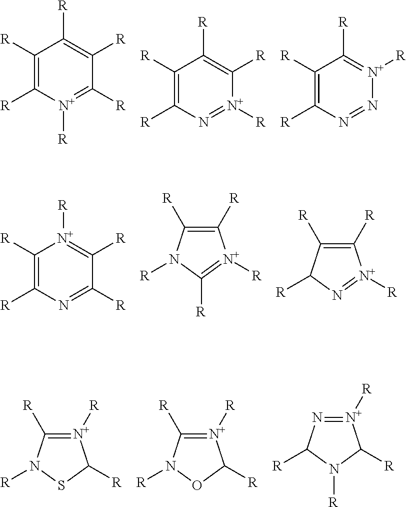

Unsaturated cyclic onium cations; in particular those selected from the group consisting of:

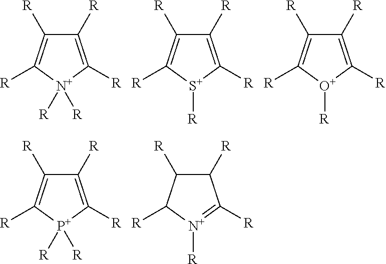

Saturated cyclic onium cations; in particular those selected from the group consisting of:

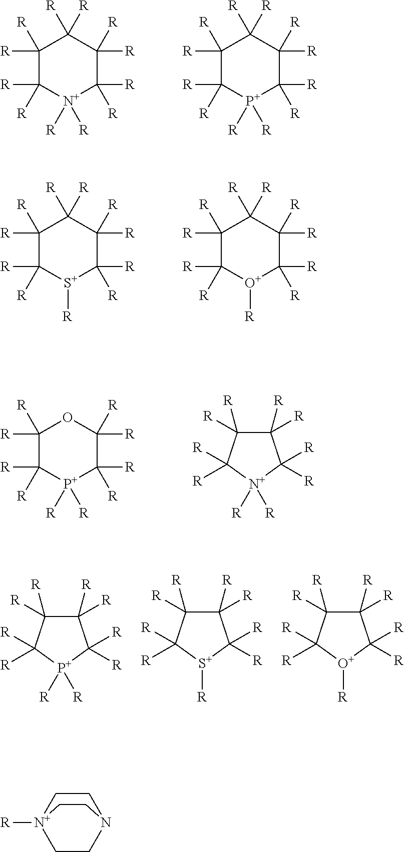

Non-cyclic onium cations; in particular those of general formula ⁺L-R′ₛ, in which L represents an atom selected from the group consisting of N, P, O and S, more preferably N and P, s represents the number of R′ groups selected from 2, 3 or 4 according to the valency of the element L, each R′ independently represents a hydrogen atom or a C₁ to C₈ alkyl group, and the bond between L⁺ and R′ can be a single bond or a double bond.

In the above formulae, each “R” symbol represents, independently of one another, a hydrogen atom or an organic group or can be bonded to one another. Preferably, each “R” symbol can represent, in the above formulae, independently of one another, a hydrogen atom or a saturated or unsaturated and linear, branched or cyclic C₁ to C₁₈ hydrocarbon group optionally substituted one or more times by a halogen atom, an amino group, an imino group, an amide group, an ether group, an ester group, a hydroxyl group, a carboxyl group, a carbamoyl group, a cyano group, a sulfone group or a sulfite group.

The onium cation Q⁺ can more particularly be selected from ammonium, phosphonium, pyridinium, pyrazolinium, imidazolium, arsenium, quaternary ammonium and quaternary phosphonium cations.

The quaternary ammonium or quaternary phosphonium cations can more preferably be selected from tetraalkylammonium or tetraalkylphosphonium cations, trialkylbenzylammonium or trialkylbenzylphosphonium cations or tetraarylammonium or tetraarylphosphonium cations, the alkyl groups of which, which are identical or different, represent a linear or branched alkyl chain having from 4 to 12 carbon atoms, preferably from 4 to 6 carbon atoms, and the aryl groups of which, which are identical or different, represent a phenyl or naphthyl group.

According to a specific embodiment, Q⁺ represents a quaternary phosphonium or quaternary ammonium cation.

According to a particularly preferred embodiment, Q⁺ represents a quaternary phosphonium cation, in particular a tetraalkylphosphonium cation and more particularly the tetrabutylphosphonium (PBu₄) cation.

According to another preferred embodiment, Q⁺ represents a quaternary ammonium cation selected in particular from the group consisting of tetraethylammonium, tetrapropylammonium, tetrabutylammonium, trimethylbenzylammonium, methyltributylammonium and Aliquat 336 (mixture of methyltri(C₈ to C₁₀ alkyl)ammonium compounds).

According to another preferred embodiment, Q⁺ represents a pyridinium cation, in particular N-methylpyridinium.

As indicated above, the Ea group can represent a fluorine atom or a group having from 1 to 10 carbon atoms selected from fluoroalkyls, perfluoroalkyls and fluoroalkenyls.

Within the context of the invention:
alkyl is understood to mean a linear or branched hydrocarbon chain preferably comprising from 1 to 10 carbon atoms, in particular from 1 to 4 carbon atoms;
fluoroalkyl is understood to mean a group formed of a linear or branched C₁-C₁₀ hydrocarbon chain comprising at least one fluorine atom;
perfluoroalkyl is understood to mean a group formed of a linear or branched C₁-C₁₀ chain comprising only fluorine atoms, in addition to the carbon atoms, and devoid of hydrogen atoms;
fluoroalkenyl is understood to mean a group formed of a linear or branched C₁-C₁₀ hydrocarbon chain comprising at least one fluorine atom and comprising at least one double bond.

The Ea group is preferably selected from a fluorine atom and a group having from 1 to 5 carbon atoms selected from fluoroalkyls, perfluoroalkyls and fluoroalkenyls.

According to a particularly preferred embodiment, the Ea group in the compound of formula (I) is selected from a fluorine atom, the CH₂F radical, the CHF₂ radical, the C₂F₅ radical and the CF₃ radical. This thus respectively results in the preparation, according to the process of the invention, of F—SOO⁻Q⁺, of CH₂F—SOO⁻Q⁺, of CHF₂—SOO⁻Q⁺, of C₂F₅—SOO⁻Q⁺ and of CF₃—SOO⁻Q⁺, where Q⁺ is as defined above.

According to a specific embodiment, Ea represents the CF₃ radical.

It is understood that the abovementioned definitions for the onium cation Q⁺ and the Ea group respectively can be combined.

Thus, according to an alternative embodiment, the process of the invention uses an ionic liquid compound of formula Ea-COO⁻Q⁺ (I), in which:
Ea is selected from a fluorine atom, the CH₂F radical, the CHF₂ radical, the C₂F₅ radical and the CF₃ radical; in particular, Ea is the CF₃ radical; and
Q⁺ represents a quaternary phosphonium cation, in particular the tetrabutylphosphonium (PBu₄) cation.

The ionic liquid compound of formula Ea-COO⁻Q⁺ (I) according to the invention can be prepared prior to its use in the sulfination reaction according to the process of the invention.

According to a first alternative embodiment, it can be obtained by reaction, preferably in the presence of an aqueous solvent, of an alkali metal salt of formula Ea-COO⁻R⁺, with R⁺ representing an alkali metal cation, for example a potassium cation, with an onium Q⁺ salt, followed, preferably, by the removal of the aqueous solvent.

According to another alternative embodiment, the ionic liquid compound of formula (I) according to the invention can be obtained by reaction, in an organic solvent, of a fluorocarboxylic acid of formula Ea-COOH or of its Ea-COO⁻ salt with an onium Q⁺ salt, followed, preferably, by the recovery of the ionic liquid compound by extraction of the organic solvent.

The onium Q⁺ salt for the preparation of the ionic liquid compound can more particularly be an onium halide, in particular an onium chloride.

The ionic liquid compound of formula (I) employed in the process of the invention can be completely or partially a recycled compound which can be obtained, for example, by separation on conclusion of the sulfination reaction or which can originate from a subsequent synthesis stage, for example by separation on conclusion of the preparation of a fluorosulfonic acid salt by oxidation.

The sulfur oxide can more particularly be sulfur dioxide.

It can be employed in the gaseous form.

A person skilled in the art is in a position to adapt the conditions for implementation, in particular in terms of temperature and of duration, of the sulfination reaction according to the invention, in order to result in the oxysulfide and fluorinated derivative in the form of a salt of formula Ea-SOO⁻Q⁺ (II).

The ionic liquid compound of formula (I) and the sulfur oxide can be employed in a sulfur oxide/compound of formula (I) molar ratio of between 0.5 and 2, in particular between 0.6 and 1.2.

The operation in which the sulfur oxide, in particular sulfur dioxide, is brought into contact with the ionic liquid compound of formula (I) can be carried out continuously, semibatchwise or batchwise.

The process according to the invention can be carried out in an apparatus making possible semibatchwise, continuous or batchwise implementation, for example in a perfectly stirred reactor, a cascade of perfectly stirred reactors which are advantageously equipped with a jacket or a tubular reactor equipped with a jacket in which a heat-exchange fluid is circulating.

According to a particularly preferred embodiment, the operation in which the sulfur oxide, in particular sulfur dioxide, is brought into contact with the ionic liquid compound of formula (I) is carried out semibatchwise.

Preferably, the sulfur oxide, in particular sulfur dioxide, is gradually added to a liquid medium, prepared beforehand, formed of the ionic liquid compound of formula (I) in the liquid state.

The sulfination reaction according to the process of the invention can be carried out by bringing the reaction medium to a temperature of between 100° C. and 200° C., in particular between 120° C. and 150° C.

The duration of the heating can be adjusted as a function of the reaction temperature selected. It can be at least one hour, in particular be between 2 hours and 5 hours.

The sulfination reaction is advantageously carried out at atmospheric pressure. Higher pressures can also be used. Thus, an absolute total pressure selected between 1 and 20 bar, preferably between 1 and 3 bar, may be suitable. According to another embodiment, the reaction can be carried out at a pressure below atmospheric pressure. The absolute total pressure can be between 1 mbar and 999 mbar, in particular between 500 mbar and 950 mbar and more particularly between 800 mbar and 900 mbar.

According to a continuous implementation, the mean residence time, which is defined as the ratio of the volume of the reaction mass to the feed flow rate, lies more particularly between 30 minutes and 10 hours, in particular between 2 hours and 4 hours.

Advantageously, the progression of the sulfination reaction can be monitored by an analytical method.

The progression of the sulfination reaction, for example the change in the concentration of oxysulfide and fluorinated derivative of formula (II) formed, can be monitored in-line (via a sampling loop, for example) or in situ by Raman spectrometry, by near infrared spectrometry or by UV spectroscopy, preferably by Raman spectrometry.

Within the context of monitoring the state of progression of the reaction by Raman spectrometry, the reactor within which the oxidation reaction takes place can be equipped with a Raman probe, connected by an optical fiber to the Raman spectrometer, said probe making it possible, for example, to monitor the concentration of compound of formula (II) in the medium.

It is preferable not to attempt to completely convert the starting ionic liquid compound of formula (I).

The progression of the sulfination reaction can be monitored by the degree of conversion of the compound of formula (I), which denotes the ratio of the molar amount of compound of formula (I) consumed during the reaction to the total amount of compound of formula (I) in the initial reaction medium. This degree can be easily calculated after assaying said compound of formula (I) remaining in the reaction medium.

Generally, the sulfination reaction is carried out up to a degree of conversion of said compound of formula (I) ranging from 10% to 90%, in particular from 30% to 70% and more particularly from 50% to 60%.

On conclusion of the sulfination reaction according to the invention, the reaction medium thus comprises a mixture of the unconsumed ionic liquid compound Ea-COO⁻Q⁺ (I) and of the oxysulfide and fluorinated derivative Ea-SOO⁻Q⁺ (II) formed.

The process of the invention can more particularly be employed in the preparation of a trifluoromethanesulfinic acid onium salt (CF₃SO₂⁻Q⁺, with Q⁺ representing an onium cation), in particular tetrabutylphosphonium trifluoromethanesulfinate (CF₃SO₂PBu₄, or tetrabutylphosphonium triflinate).

The latter can advantageously be used to access, for example, lithium bis(trifluoromethanesulfonyl)imide (CF₃SO₂)₂NLi (LiTFSI), triflic acid CF₃SO₃H or also triflic anhydride (CF₃—SO₂)₂O, as described in detail in the continuation of the text.

The oxysulfide and fluorinated compound of formula Ea-SOO⁻Q⁺ (II) can be used to form, by an oxidation reaction, a compound of formula Ea-SO₃⁻Q⁺ (III).

The present invention thus relates, according to another of its aspects, to a process for the preparation of a compound in the form of a salt of formula (III):

Ea-SO₃⁻Q⁺ (III)
Ea representing a fluorine atom or a group having from 1 to 10 carbon atoms selected from fluoroalkyls, perfluoroalkyls and fluoroalkenyls; and
Q⁺ representing an onium cation,

comprising the stages consisting in:

(i) having available a mixture (M), exhibiting a melting point of less than or equal to 100° C., of an ionic liquid compound of formula Ea-COO⁻Q⁺ (I) and of a compound of formula Ea-SOO⁻Q⁺ (II); and

(ii) bringing together said mixture (M) in the liquid state and an oxidizing agent, in order to obtain the compound of formula (III), said mixture (M) representing more than 50% by weight of the initial liquid reaction medium.

The mixture (M) of stage (i) can be the liquid reaction mixture directly obtained on conclusion of the sulfination stage described above, the sulfination reaction being more particularly carried out up to a degree of conversion of said ionic liquid compound of formula (I) ranging from 10% to 90%, in particular from 30% to 70% and more particularly from 50% to 60%.

According to a particularly advantageous embodiment, the sulfination and oxidation stages can be linked together, for example within one and the same semibatchwise reactor, without requiring an operation for change of solvent.

The compound of formula Ea-SO₃⁻Q⁺ (III), where Ea and Q⁺ are as defined above, can thus be prepared according to the invention via the following stages:

(a) bringing an ionic liquid compound of formula Ea-COO⁻Q⁺ (I) in the liquid state, where Q represents an onium cation, into contact with a sulfur oxide, in order to form a compound of formula Ea-SOO⁻Q⁺ (II), said ionic liquid compound of formula (I) representing at least 50% by weight of the initial liquid reaction medium;

the sulfination reaction being carried out up to a degree of conversion of said compound of formula (I) ranging from 10% to 90%, in particular from 30% to 70% and more particularly from 50% to 60%;

(b) addition of an oxidizing agent to the liquid reaction mixture (M) of the compounds of formulae Ea-COO⁻Q⁺ (I) and Ea-SOO⁻Q⁺ (II) obtained on conclusion of the sulfination stage (a), in order to form a compound of formula Ea-SO₃⁻Q⁺ (III).

The mixture (M) of the compounds of formulae Ea-COO⁻Q⁺ (I) and Ea-SOO⁻Q⁺ (II) can more particularly exhibit a melting point of less than or equal to 80° C., in particular of less than or equal to 40° C. and preferably of less than or equal to 20° C.

The oxysulfide and fluorinated derivative of formula Ea-SOO⁻Q⁺ (II) and the ionic liquid compound of formula Ea-COO⁻Q⁺ (I) can be present within the mixture (M) in a compound (II)/compound (I) ratio by weight of between 0.2 and 4, in particular between 0.5 and 3.

Of course, the composition of the mixture (M), formed on conclusion of the sulfination process according to the invention described above, depends on the degree of conversion of the compound of formula (I) on conclusion of the sulfination reaction.

Advantageously, the oxidation reaction according to the invention does not require the addition of an ancillary solvent.

In particular, the reaction medium of the oxidation reaction according to the invention is devoid of aqueous solvent.

The absence of aqueous solvent does not exclude the possible presence of water in a low content unsuited to the dissolution of a reactant.

Thus, the reaction medium of the oxidation reaction according to the invention can comprise a water content of less than or equal to 20% by weight, in particular of less than or equal to 10% by weight.

These small amounts of water can more particularly originate from the oxidizing agent employed in the oxidation reaction, for example aqueous hydrogen peroxide, and/or be formed by the oxidation reaction.

The mixture of the compounds of formulae Ea-COO⁻Q⁺ (I) and Ea-SOO⁻Q⁺ (II), in the liquid state under the conditions of the oxidation reaction, acts both as reactant and as solvent for the oxidation reaction.

The mixture, denoted (M), of the compounds of formulae Ea-COO⁻Q⁺ (I) and Ea-SOO⁻Q⁺ (II) thus represents at least 50% by weight of the initial liquid reaction medium.

In the specific case of the batchwise implementation of the oxidation reaction, the initial reaction medium comprises said mixture (M) and the whole of the amount of oxidizing agent employed in the oxidation reaction. The liquid mixture (M) according to the invention can represent from 50% to 95% by weight of the initial reaction medium.

In the specific case of the semibatchwise implementation of the oxidation reaction, the oxidizing agent can be added gradually. The liquid mixture (M) according to the invention can represent from 50% to 100% by weight of the initial reaction medium.

According to a particularly preferred embodiment, the initial reaction medium of the oxidation stage according to the invention is formed of said mixture (M) of the compounds of formulae Ea-COO⁻Q⁺ (I) and Ea-SOO⁻Q⁺ (II), and optionally of the oxidizing agent, according to whether the oxidation reaction is carried out batchwise, semibatchwise or continuously.

The oxidizing agent can be selected from peroxides, peracids and their salts. For example, the oxidizing agent can be selected from aqueous hydrogen peroxide; percarbonates, in particular sodium or potassium percarbonate; persulfates, in particular potassium persulfate; persulfuric acid, for example Caro's salt; and organic peroxides, for example urea-hydrogen peroxide. The oxidizing agent can also be sodium hypochlorite.

The oxidizing agent can be miscible or immiscible in the reaction medium. Thus, the reaction medium can be homogeneous or heterogeneous.

According to a particularly advantageous embodiment, the oxidizing agent is anhydrous.

According to another specific embodiment, the oxidizing agent is aqueous hydrogen peroxide. The aqueous hydrogen peroxide can have a concentration in water of between 10% and 80%, preferably between 30% and 70%.

Moreover, the oxidizing agent can be selected from gaseous agents, for example from the group consisting of air, oxygen, (O₂), ozone (O₃) and nitrous oxide (N₂O). Oxidation with these agents can optionally be carried out in the presence of a metal catalyst.

A person skilled in the art is in a position to adapt the conditions for carrying out the oxidation reaction in order to result in the desired compound of formula (III).

The liquid mixture (M) can be brought into contact with the oxidizing agent continuously, semibatchwise or batchwise. They are preferably brought into contact semibatchwise. They can be brought into contact in an apparatus as described above for the sulfination process according to the invention.

According to a particularly preferred semibatchwise embodiment, the oxidizing agent, for example aqueous hydrogen peroxide, is gradually added to the liquid mixture (M).

The oxidation reaction according to the process of the invention can be carried out by bringing the reaction medium to a temperature of between 20° C. and the boiling point of the organic solvent, in particular between 40° C. and 140° C. Advantageously, the oxidizing agent can be added after having preheated the mixture (M).

The duration of the heating can be adjusted as a function of the reaction temperature selected. It can be between 30 minutes and 24 hours, in particular between 1 hour and 20 hours and more particularly between 2 hours and 7 hours.

As for the sulfination stage described above, the progression of the oxidation reaction can be monitored by an analytical method as described above, preferably by Raman spectrometry.

On conclusion of the oxidation reaction according to the invention, the reaction medium is formed essentially of the mixture of the ionic liquid compound of formula Ea-COO⁻Q⁺ (I) and of the compound of formula Ea-SO₃⁻Q⁺ (III) formed.

Advantageously, the oxidation process according to the invention is employed in order to prepare a trifluoromethanesulfonic acid onium salt (CF₃SO₃⁻Q⁺, with Q⁺ representing an onium cation), in particular tetrabutylphosphonium trifluoromethanesulfonate (CF₃SO₃PBu₄, or tetrabutylphosphonium triflate).

The latter can advantageously be used to access triflic acid (CF₃SO₃H) or also triflic anhydride ((CF₃SO₂)₂0), as described in detail in the continuation of the text.

The oxysulfide and fluorinated derivatives of formula Ea-SO₂⁻Q⁺ (II) obtained according to the sulfination process of the invention described above can advantageously be used in the synthesis of sulfonimide compounds of formula (Ea-SO₂)₂NH (IV) and of their salts (Ea-SO₂)₂NMe (IV′), with Me representing an alkali metal.

According to another of its aspects, the invention thus relates to a process for the preparation of a sulfonimide compound (Ea-SO₂)₂NH (IV) or one of its salts (Ea-SO₂)₂NMe (IV′),

Ea representing a fluorine atom or a group having from 1 to 10 carbon atoms selected from fluoroalkyls, perfluoroalkyls and fluoroalkenyls; and

Me representing an alkali metal, in particular lithium;

comprising at least the following stages:

(a1) preparation of an oxysulfide and fluorinated derivative of formula Ea-SO₂⁻Q⁺ (II) according to the process described above;

(b1) bromination, chlorination or fluorination of the derivative of formula (II) in order to form a compound of formula (Ea-SO₂)X, with X representing bromine, chlorine or fluorine;

(c1) ammonolysis of the compound (Ea-SO₂)X using a tertiary amine NR″₃ to give (EaSO₂)₂NH.NR″₃, with R″, which are identical or different, representing a linear or branched alkyl group having from 1 to 20 carbon atoms;

(d1) acidification of (EaSO₂)₂NH.NR″₃ to obtain the sulfonimide compound (Ea-SO₂)₂NH (IV);

and optionally:

(e1) neutralization by an alkali metal Me base, in particular by an alkali metal hydroxide, of the compound (Ea-SO₂)₂NH in order to form the salt of formula (Ea-SO₂)₂NMe (IV′); and optionally

(f1) drying of the salt (Ea-SO₂)₂NMe (IV′).

The tertiary amine employed in the ammonolysis stage (c1) can, for example, be diisopropylethylamine (EDIPA).

According to another of its aspects, another subject matter of the present invention is the linking together of the stages (a1) and (b1) described above. Thus, the invention relates to a process for the preparation of a compound (Ea-SO₂)X, with X representing chlorine or fluorine, comprising:
the preparation of an oxysulfide and fluorinated derivative of formula Ea-SO₂⁻Q⁺ (II) according to the process described above; and
the bromination, chlorination or fluorination of the derivative of formula (II) in order to form a compound of formula (Ea-SO₂)X, with X representing bromine, chlorine or fluorine.

According to a particularly preferred embodiment, the oxysulfide and fluorinated derivative of formula (II) is a trifluoromethanesulfinic acid onium salt CF₃SO₂⁻Q⁺, for example tetrabutylphosphonium trifluoromethanesulfinate (CF₃SO₂PBu₄), in order to access, according to the process described above, bis(trifluoromethanesulfonyl)imide (CF₃SO₂)₂NH and lithium bis(trifluoromethanesulfonyl)imide (CF₃SO₂)₂NLi (LiTFSI).

According to another particularly preferred embodiment, the oxysulfide and fluorinated derivative of formula (II) is a fluorosulfinic acid onium salt F—SO₂⁻Q⁺, for example tetrabutylphosphonium fluorosulfinate (F—SO₂PBu₄), in order to access, according to the process described above, bis(fluorosulfonyl)imide (F—SO₂)₂NH and lithium bis(fluorosulfonyl)imide (F—SO₂)₂NLi (LiFSI).

The sulfonimide compounds and their salts prepared according to the process described above can advantageously be used as electrolyte salts, as antistatic agent precursors or as surfactant precursors. In particular, said compounds can advantageously be employed as electrolytes in the manufacture of batteries, in the fields of electrochromism, electronics and electrochemistry. They are advantageously employed as antistatic agents in the manufacture of pressure-sensitive adhesives (PSAs). As antistatic agents, they can also be employed as components of lubricants. They are used in optical materials, such as electroluminescent devices, and participate in the composition of photovoltaic panels. These uses are also subject matters of the invention. In particular, a subject matter of the invention is a process for the manufacture of an electrochemical device, preferably a battery, said process comprising a stage of preparation of a sulfonimide compound or of its salts according to the process described above and a stage of manufacture of the electrochemical device in which the sulfonimide compound or its salts is employed as electrolyte.

Advantageously, the derivatives of formula Ea-SO₃⁻Q⁺ (III) obtained according to the invention can be employed in the preparation of fluorinated derivatives of sulfonic acid Ea-SO₃H.

Thus, according to yet another of its aspects, a subject matter of the invention is a process for the preparation of a fluorinated derivative of sulfonic acid of formula (V):

Ea-SO₃H (V)
Ea representing a fluorine atom or a group having from 1 to 10 carbon atoms selected from fluoroalkyls, perfluoroalkyls and fluoroalkenyls, in particular Ea representing the CF₃ radical;
   comprising at least the following stages:
preparation according to the process described above of a compound of formula Ea-SO₃⁻Q⁺ (III), in which Q⁺ represents an onium cation; and
acidification of the compound of formula (III) in order to obtain the desired fluorinated derivative of sulfonic acid of formula (V).

In particular, a fluorinated derivative of sulfonic acid of formula Ea-SO₃H (V), where Ea is as defined above, can be prepared according to the invention via at least the following stages:

(a) bringing an ionic liquid compound of formula Ea-COO⁻Q⁺ (I) in the liquid state, where Q represents an onium cation, into contact with a sulfur oxide, in order to form a compound of formula Ea-SOO⁻Q⁺ (II), said ionic liquid compound of formula (I) representing at least 50% by weight of the initial liquid reaction medium;

the sulfination reaction being carried out up to a degree of conversion of said compound of formula (I) ranging from 10% to 90%, in particular from 30% to 70% and more particularly from 50% to 60%;

(b) addition of an oxidizing agent to the liquid reaction mixture (M) of the compounds of formulae Ea-COO⁻Q⁺ (I) and Ea-SOO⁻Q⁺ (II) obtained on conclusion of the sulfination stage (a), in order to form a compound of formula Ea-SO₃⁻Q⁺ (III); and

(c2) acidification in order to obtain the desired fluorinated derivative of sulfonic acid of formula (V).

The stages of sulfination (a) and of oxidation (b) are more particularly carried out under the conditions described above.

The acidification of the compound of formula Ea-SO₃⁻Q⁺ (III) can be carried out by addition of sulfuric acid, in particular in the oleum form.

More particularly, the acidification of the mixture of the compounds of formulae Ea-SO₃⁻Q⁺ and Ea-COO⁻Q⁺, obtained on conclusion of the oxidation, results in the mixture of the desired fluorinated derivative of sulfonic acid Ea-SO₃H and of the fluorocarboxylic acid Ea-COOH, for example in the mixture of triflic acid and trifluoroacetic acid (in the specific case where Ea represents CF₃).

The fluorinated derivative of sulfonic acid Ea-SO₃H can be isolated from the mixture obtained on conclusion of the acidification, for example by distillation.

The fluorinated derivative of carboxylic acid Ea-COOH is advantageously recycled, for example in the process according to the invention.

Advantageously, the process of the invention is employed to prepare trifluoromethanesulfonic acid CF₃SO₃H, more commonly known as triflic acid.

According to a specific embodiment, the compound of formula (I) employed in stage (a) is a trifluorocarboxylic acid onium salt, in particular tetrabutylphosphonium trifluorocarboxylate (CF₃COOPBu₄), and results, on conclusion of stage (c2), in triflic acid (CF₃SO₃H).

The fluorinated derivative of sulfonic acid Ea-SO₃H obtained according to the invention can advantageously be converted into the anhydride of formula (Ea-SO₂)₂O (VI).

Thus, according to yet another of its aspects, a subject matter of the invention is a process for the preparation of an anhydride compound of formula (Ea-SO₂)₂O (VI), Ea representing a fluorine atom or a group having from 1 to 10 carbon atoms selected from fluoroalkyls, perfluoroalkyls and fluoroalkenyls, Ea in particular representing the CF₃ radical; comprising at least the following stages:
preparation, according to the process described above, of a fluorinated derivative of sulfonic acid of formula Ea-SO₃H (V); and
anhydrization of the derivative of formula Ea-SO₃H (V) in order to obtain said desired anhydride compound of formula (VI).

In particular, an anhydride compound of formula (Ea-SO₂)₂O (VI), where Ea is as defined above, can be prepared according to the invention via at least the following stages:

(a) bringing an ionic liquid compound of formula Ea-COO⁻Q⁺ (I) in the liquid state, where Q represents an onium cation, into contact with a sulfur oxide, in order to form a compound of formula Ea-SOO⁻Q⁺ (II), said ionic liquid compound of formula (I) representing at least 50% by weight of the initial liquid reaction medium;

the sulfination reaction being carried out up to a degree of conversion of said compound of formula (I) ranging from 10% to 90%, in particular from 30% to 70% and more particularly from 50% to 60%;

(b) addition of an oxidizing agent to the liquid reaction mixture (M) of the compounds of formulae Ea-COO⁻Q⁺ (I) and Ea-SOO⁻Q⁺ (II) obtained on conclusion of the sulfination stage (a), in order to form a compound of formula Ea-SO₃⁻Q⁺ (III);

(c2) acidification in order to obtain the fluorinated derivative of sulfonic acid of formula Ea-SO₃H (V); and

(d3) anhydrization of the compound of formula (V) in order to form the desired anhydride compound of formula (VI).

The stages of sulfination (a), of oxidation (b) and of acidification (c2) are more particularly carried out under the conditions described above.

The anhydrization reaction is known to a person skilled in the art and is more particularly described in the document U.S. Pat. No. 8,222,450.

Advantageously, the process of the invention is employed to prepare trifluoromethanesulfonic anhydride (CF₃—SO₂)₂O, more commonly known as triflic anhydride.

According to a specific embodiment, the compound of formula (I) employed in stage (a) is a trifluorocarboxylic acid onium salt, in particular tetrabutylphosphonium trifluorocarboxylate (CF₃COOPBu₄), and results, on conclusion of stage (d3), in triflic anhydride ((CF₃—SO₂)₂O).

The fluorinated derivatives of sulfonic acid of formula Ea-SO₃H, in particular triflic acid, and the anhydride compounds of formula (Ea-SO₂)₂O, in particular triflic anhydride, can be used in various applications, in particular as acid catalyst, as protective group in organic synthesis, as synthon in the fields of pharmaceuticals, agrochemistry or electronics, as salt for the electronics industry or as component of an ionic liquid.

The invention will now be described by means of the following examples, of course given by way of nonlimiting illustration of the invention.

### EXAMPLES

The degree of conversion of a reactant corresponds to the ratio of the molar amount of reactant consumed (converted) during a reaction to the initial amount of reactant.

The product yield from a reactant corresponds to the ratio of the molar amount of product formed to the molar amount of initial reactant.

The weight balance corresponds to the ratio of the total weight of material recovered to the total weight of material charged.

1. Preparation of TFAPBu₄ (CF₃CO₂PBu₄)

The following are introduced into a 0.25 l glass reactor:
TFAK (CF₃COOK): 43.8 g (˜0.3 mol)
PBu₄Cl: 86.7 g (˜0.3 mol)
H₂O: 110 g The reaction mass is brought to 50° C. with stirring for 20 hours. After cooling and halting the stirring, the medium separates on settling into two liquid phases, which are separated and analyzed; the upper phase is essentially composed of CF₃CO₂PBu₄ and of water (7% by weight).

The reaction for the formation of TFAPBu₄ can be represented as follows:

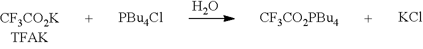

2. Sulfination of TFAPBu₄ to Give TFSPBu₄ (CF₃SO₂PBu₄)

The following are introduced into a 0.1 ml autoclave which has been washed and dried beforehand:
TFAPBu₄: 49.2 g, i.e. 0.12 mol (dried beforehand by azeotropic distillation),
SO₂: 7.76 g, i.e. SO₂/TFAPBu₄ 103 mol %.

The reactor is subsequently closed and heated with stirring at 142° C. for 4 h.

After returning to ambient temperature (20-22° C.), the reactor is degassed and its contents are transferred into a 0.1 l glass vessel; the resulting reaction medium exists in the form of a dark brown solution.

The reaction for the sulfination of TFAPBu₄ to give TFSPBu₄ can be represented as follows:

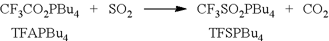

The analysis by ¹⁹F NMR of the reaction mass gives the following results:
Weight balance: 94%
Conversion of the TFAPBu₄: 61%
Yield of TFSPBu₄: 38%

3. Oxidation of the Triflinate CF₃SO₂PBu₄ (TFSPBu₄) to Give the Triflate CF₃SO₃PBu₄ (TAPBu₄)

The crude reaction product from the preceding stage 2 (45 g) is charged to a 0.1 l three-necked round-bottomed flask and brought to 80° C.; the aqueous hydrogen peroxide (30% aqueous solution: 6 g) is run, over 3 hours, onto the reaction medium maintained 80° C.

After the aqueous hydrogen peroxide has been run onto the reaction medium, the latter is maintained at 80° C. for 2 hours.

After returning to ambient temperature (20-22° C.), the resulting reaction medium exists in the form of a light brown solution.

The reaction for the oxidation of the triflinate to give the triflate can be represented as follows:

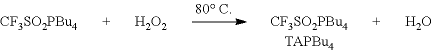

The analysis by ¹⁹F NMR of the reaction mass gives the following results:
Weight balance: 100%
Conversion of the TFSPBu₄: 100%
Yield of TAPBu₄: 97%

NB: conversion of the residual TFAPBu₄: 0%

4. Acidification/Distillation

The following are introduced into a 50 ml glass reactor:
preceding crude reaction product: 50 g
20% oleum: 122 g

The medium is gradually brought to 160° C. with stirring under gradual vacuum (final pressure: 5 mbar) and the condensates are collected and analyzed to give the following results:
Degree of recovery of the TFA (CF₃COOH): 65%
Degree of recovery of the TA (CF₃SO₃H): 50%

1. A process for the preparation of an oxysulfide and fluorinated derivative in the form of a salt of formula (II):
Ea-SOO⁻Q⁺ (II)
the process comprising the operation in which an ionic liquid compound of formula (I) in the liquid state:
Ea-COO⁻Q⁺ (I)
Ea representing a fluorine atom or a group having from 1 to 10 carbon atoms selected from the group consisting of fluoroalkyls, perfluoroalkyls and fluoroalkenyls; and
Q⁺ representing an onium cation,
is brought together with a sulfur oxide, said ionic liquid compound of formula (I) representing at least 50% by weight of the initial liquid reaction medium.

2. The process as claimed in claim 1, in which the reaction medium is devoid of organic solvent of amide type.

3. The process as claimed in claim 1, in which the onium cation Q⁺ is selected from the group consisting of ammonium, phosphonium, pyridinium, pyrazolinium, imidazolium, arsenium, quaternary ammonium and quaternary phosphonium cations.

4. The process as claimed in claim 1, in which the onium cation Q⁺ is a quaternary phosphonium cation.

5. The process as claimed in claim 1, in which the cation Q⁺ represents a quaternary ammonium cation selected from the group consisting of tetraethylammonium, tetrapropylammonium, tetrabutylammonium, trimethylbenzylammonium, methyltributylammonium and Aliquat 336.

6. The process as claimed in claim 1, in which the cation Q⁺ represents a pyridinium cation.

7. The process as claimed in claim 1, for the preparation of a trifluoromethanesulfinic acid onium salt CF ₃SOO⁻Q⁺.

8. The process as claimed in claim 7, in which the trifluoromethanesulfinic acid onium salt CF₃SOO⁻Q⁺ is tetrabutylphosphonium trifluoromethanesulfinate CF₃SOOPBu₄.

9. A process for the preparation of a compound in the form of a salt of formula (III):
Ea-SO₃⁻Q⁺ (III)
Ea representing a fluorine atom or a group having from 1 to 10 carbon atoms selected from the group consisting of fluoroalkyls, perfluoroalkyls and fluoroalkenyls; and
Q⁺ representing an onium cation,
the process comprising:
(i) having available a mixture (M), exhibiting a melting point of less than or equal to 100° C., of an ionic liquid compound of formula Ea-COO⁻Q⁺ (I) and of a compound of formula Ea-SOO⁻Q⁺ (II); and
(ii) bringing together said mixture (M) in the liquid state and an oxidizing agent, in order to obtain the compound of formula Ea-SO₃⁻Q⁺ (III), said mixture (M) representing more than 50% by weight of the initial liquid reaction medium.

10. The process as claimed in claim 9, for the preparation of a trifluoromethanesulfonic acid onium salt CF₃SO₃⁻Q⁺.

11. The process as claimed in claim 10, in which the trifluoromethanesulfonic acid onium salt CF₃SO₃⁻Q⁺ is tetrabutylphosphonium trifluoromethanesulfonate CF₃SO₃PBu₄.

12. A process for the preparation of a sulfonimide compound of formula (Ea-SO₂)₂NH (IV) or one of its salts (Ea-SO₂)₂NMe (IV′),
Ea representing a fluorine atom or a group having from 1 to 10 carbon atoms selected from the group consisting of fluoroalkyls, perfluoroalkyls and fluoroalkenyls; and
Me representing an alkali metal;
the process comprising at least the following stages:
(a1) preparation of an oxysulfide and fluorinated derivative of formula Ea-SO₂⁻Q⁺ (II) according to the process described in claim 1;
(b1) bromination, chlorination or fluorination of the derivative of formula (II) in order to form a compound of formula (Ea-SO₂)X, with X representing bromine, chlorine or fluorine;
(c1) ammonolysis of the compound (Ea-SO₂)X using a tertiary amine NR″₃ to give (EaSO₂)₂NH.NR″₃, with R″, which are identical or different, representing a linear or branched alkyl group having from 1 to 20 carbon atoms;
(d1) acidification of (EaSO₂)₂NH.NR″₃ to obtain the sulfonimide compound (Ea-SO₂)₂NH (IV);
and optionally:
(e1) neutralization by an alkali metal Me base of the compound (Ea-SO₂)₂NH in order to form the salt of formula (Ea-SO₂)₂NMe (IV′); and optionally
(f1) drying of the salt (Ea-SO₂)₂NMe (IV′).

13. The process as claimed in claim 12, for the preparation of bis(trifluoromethanesulfonyl)imide (CF₃SO₂)₂NH and of lithium bis(trifluoromethanesulfonyl)imide (LiTFSI).

14. A process for the preparation of a fluorinated derivative of sulfonic acid of formula (V):
Ea-SO₃H (V)
Ea representing a fluorine atom or a group having from 1 to 10 carbon atoms selected from the group consisting of fluoroalkyls, perfluoroalkyls and fluoroalkenyls;
the process comprising at least the following stages:
preparation according to the process as defined in claim 9 of a derivative of formula Ea-SO₃⁻Q⁺ (III), in which Q⁺ represents an onium cation; and
acidification of the compound of formula (III) in order to obtain the desired fluorinated derivative of sulfonic acid of formula (V).

15. The process as claimed in claim 14, for the preparation of trifluoromethanesulfonic acid CF₃50₃H.

16. A process for the preparation of an anhydride compound of formula (VI):
(Ea-SO₂)₂O (VI)
Ea representing a fluorine atom or a group having from 1 to 10 carbon atoms selected from the group consisting of fluoroalkyls, perfluoroalkyls and fluoroalkenyls;
the process comprising at least the following stages:
preparation according to the process as defined according to claim 14 of a fluorinated derivative of sulfonic acid of formula Ea-SO₃H; and
anhydrization of the derivative of formula Ea-SO₃H in order to obtain said desired anhydride compound of formula (VI).

17. The process as claimed in claim 16, for the preparation of trifluoromethanesulfonic anhydride (CF₃—SO₂)₂O.

18. The process as claimed in claim 3, in which the onium cation Q⁺ is selected from the group consisting of tetraalkylphosphonium and tetraalkylammonium cations, the alkyl groups of which, which are identical or different, represent a linear or branched alkyl chain having from 4 to 12 carbon atoms, and tetraarylphosphonium and tetraarylammonium cations, the aryl groups of which, which are identical or different, represent a phenyl or naphthyl group.

19. The process as claimed in claim 4, in which the onium cation Q⁺ is tetrabutylphosphonium (PBu₄) cation.

20. The process according to claim 9, wherein the mixture (M) exhibits a melting point of less than or equal to 40° C.
